(19) United States
(12) Patent Application Publication
(10) Pub. No.: US 2023/0241370 A1
(43) Pub. Date: Aug. 3, 2023

(54) Multifunction Valvuloplasty Catheter

(71) Applicant: Ethan Chauncey Korngold, Portland, OR (US)

(72) Inventor: Ethan Chauncey Korngold, Portland, OR (US)

(21) Appl. No.: 18/105,073

(22) Filed: Feb. 2, 2023

Related U.S. Application Data
(60) Provisional application No. 63/305,688, filed on Feb. 2, 2022.

(51) Int. Cl. A61M 60/13; A61M 60/857

(52) U.S. Cl. CPC A61M 60/13; A61M 60/857

(57) ABSTRACT

A multifunction valvuloplasty catheter device and a method of using the catheter device is provided that serves to streamline the aortic valvuloplasty procedure and reduce opportunities for error and complications by minimizing catheter exchanges. In one aspect, a multifunction catheter is adapted for valvuloplasty. The multifunction catheter comprises a shaft defining at least one lumen. The shaft includes a distal end, a proximal end, and a bend. A valvuloplasty expansion device comprises a balloon or expandable braid valvuloplasty device, and the valvuloplasty expansion device is disposed along the shaft between the proximal end and the bend of the shaft. An opening through the shaft or a pressure sensor is provided on the catheter along the shaft between the bend and the distal end of the shaft. The opening or pressure sensor is adapted to facilitate a hemodynamic pressure measurement within a left ventricle of a heart.

### CROSS-REFERENCE TO RELATED APPLICATIONS

This application claims the benefit of United States provisional application no. 63/305,688, filed Feb. 2, 2022, which is hereby incorporated by reference as though fully set forth herein.

### BACKGROUND

### A. Field

The instant disclosure relates to a multi-function valvuloplasty catheter device adapted to treat aortic valve stenosis. In particular, the multifunction valvuloplasty catheter device is adapted to facilitate crossing of the aortic valve with a guidewire, measure hemodynamics, and perform aortic valvuloplasty.

### B. Background

Aortic stenosis is a common and serious heart valve condition that involves narrowing of the aortic valve. One technique for treating aortic stenosis is balloon aortic valvuloplasty, where a balloon is delivered through the arterial system over a guidewire into the stenotic aortic valve, then inflated and deflated to expand the valve orifice. In order to perform the valvuloplasty procedure, the narrowed aortic valve is crossed with a guidewire within a shaped catheter. After wire crossing, a common practice is to remove the initial catheter and place a second catheter, such as a pigtail catheter over the guidewire across the aortic valve into the left ventricle to measure hemodynamic pressure gradients across the aortic valve. This catheter has the characteristic that it is atraumatic to the left ventricle. Aortic valvuloplasty is often performed by removing this catheter and placing a balloon catheter into the aortic valve and inflating and deflating the balloon. If desired, the atraumatic catheter is replaced to measure hemodynamics and assess the hemodynamic effect of the valvuloplasty.

Crossing the stenotic aortic valve with a guidewire is often technically challenging and requires time and skill, since the aortic valve is narrowed, and the guidewire is being advanced against the direction of the blood flow. To support the guidewire and position it near the aortic valve orifice to enable guidewire crossing, a shaped catheter is employed. These shaped catheters (e.g., AL1 or JR4 catheters) are typically designed for engagement of the coronary arteries and not specifically designed for the purpose of crossing the aortic valve. Once the guidewire is across the aortic valve, the catheter is exchanged for an atraumatic catheter and then a balloon catheter. These catheter exchanges require extra materials and time during the procedure. Any catheter exchange requires passage of equipment through the arterial system and across the aortic valve and carries a risk of trauma to the arterial system and and/or embolization of debris resulting in complications. It is noted that currently utilized balloon valvuloplasty catheters designed for aortic valvuloplasty are not designed to facilitate crossing with a guidewire, to be atraumatic, or to measure hemodynamics.

### BRIEF SUMMARY

A multifunction valvuloplasty catheter device and a method of using the catheter device is provided that serves to streamline the aortic valvuloplasty procedure and reduce opportunities for error and complications by minimizing catheter exchanges.

In one aspect, a multifunction catheter is adapted for valvuloplasty. The multifunction catheter comprises a shaft defining at least one lumen. The shaft includes a distal end, a proximal end, and a bend. A valvuloplasty expansion device comprises a balloon or expandable braid valvuloplasty device, and the valvuloplasty expansion device is disposed along the shaft between the proximal end and the bend of the shaft. An opening through the shaft or a pressure sensor is provided on the catheter along the shaft between the bend and the distal end of the shaft. The opening or pressure sensor is adapted to facilitate a hemodynamic pressure measurement within a left ventricle of a heart.

In one aspect, the bend of the multifunction catheter shaft comprises at least one of the group comprising: a single bend, a double bend, and a pigtail bend.

In another aspect, a pressure sensor comprises at least one of an electrical and mechanical pressure sensor adapted to facilitate a hemodynamic measurement within a left ventricle of a heart.

In another aspect, an atraumatic tip is disposed at the distal end of the shaft.

In another aspect, a balloon comprises a wrapped and pleated material disposed along a length of the shaft.

In another aspect, at least one port is provided at the shaft in fluid communication with at least one lumen.

In another aspect, a valvuloplasty expansion device comprises an expandable braid valvuloplasty device. The expandable braid valvuloplasty device includes a plurality of interwoven wires surrounding the at least one lumen defined by the shaft, and the plurality of interwoven wires are fixed to the shaft distal to an expansion location of the valvuloplasty expansion device.

In another aspect, a method for performing a valvuloplasty procedure on a patient is provided. The method comprises crossing distal ends of a guidewire and a multifunction catheter across an aortic valve orifice and into a left ventricle of a heart. In various aspects, the guidewire and catheter may be inserted into the arterial system of a patient together and directed to the aortic valve orifice, or the guidewire may be inserted into the patient arterial system first, and then the catheter is directed along the guidewire to the aortic valve orifice. A hemodynamic pressure measurement is performed within the left ventricle of the heart via an opening or pressure sensor disposed along a shaft of the multifunction catheter. A balloon or an expandable braid valvuloplasty expansion device is expanded within the aortic valve orifice. The balloon or the expandable braid valvuloplasty expansion device is disposed proximal to the opening or pressure sensor along the shaft of the multifunction catheter. Another hemodynamic pressure measurement is performed within the left ventricle of the heart via the opening or pressure sensor of the multifunction catheter. It should be noted that neither measurement operation, one measurement operation, or both measurement operations may be performed in various implementations. The guidewire and multifunction catheter are withdrawn from the patient. Again, the guidewire and catheter may be withdrawn together or separately.

In one aspect of the method, a multifunction catheter used in the method comprises a shaft defining at least one lumen. The shaft includes a distal end, a proximal end, and a bend. A valvuloplasty expansion device comprises a balloon or expandable braid valvuloplasty device, and the valvuloplasty expansion device is disposed along the shaft between the proximal end and the bend of the shaft. An opening through the shaft or a pressure sensor is provided on the catheter along the shaft between the bend and the distal end of the shaft. The opening or pressure sensor is adapted to facilitate a hemodynamic pressure measurement within a left ventricle of a heart.

In another aspect, a second hemodynamic pressure measurement is performed after expanding the balloon or the expandable braid valvuloplasty expansion device within the aortic valve orifice.

In another aspect, the guidewire and the multifunction catheter are advanced through the aortic valve orifice together.

In another aspect, the guidewire is advanced through the aortic valve orifice followed by the multifunction catheter being advanced along the guidewire.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a schematic diagram of a multifunction valvuloplasty catheter device optimized for positioning a guidewire to cross the aortic valve, atraumatic advancement and positioning of the device in the left ventricle, measurement of hemodynamics, and aortic valvuloplasty.

FIG. 2 is a schematic diagram of another representative embodiment of a multifunction valvuloplasty catheter device with a different shaped catheter adapted to cross the aortic valve.

FIG. 3 is a schematic diagram of yet another representative embodiment of a multifunction valvuloplasty catheter device with a pigtail configuration.

FIG. 4 is a schematic diagram of another representative embodiment of a multifunction catheter device with a pressure sensor.

FIGS. 5 to 13 are schematic diagrams showing example transcatheter aortic valve replacement procedure (TAVR) procedure. This multifunction catheter may be used as part of a TAVR procedure, or as a standalone balloon aortic valvuloplasty not accompanied by TAVR, or as part of another procedure

FIGS. 14 is a flow chart showing operations of an example method of using a multifunction catheter as described herein.

FIG. 15A and 15B are schematic diagrams of yet another representative embodiment of a multifunction catheter device having a mechanical expandable braid valvuloplasty device comprising a plurality of interwoven wires.

### DETAILED DESCRIPTION

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used throughout, the singular forms “a,” “an” and “the” include plural referents unless the context clearly dictates otherwise. Thus, for example, reference to “a” component can include two or more such components unless the context indicates otherwise. Also, the words “proximal” and “distal” are used to describe items or portions of items that are situated closer to and away from, respectively, a user or operator such as a surgeon. Thus, for example, the tip or free end of a device may be referred to as the distal end, whereas the generally opposing end or handle may be referred to as the proximal end.

All directional references (e.g., upper, lower, upward, downward, left, right, leftward, rightward, top, bottom, above, below, vertical, horizontal, clockwise, and counterclockwise) are only used for identification purposes to aid the reader’s understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

The term “substantially” as used herein may be applied to modify any quantitative representation which could permissibly vary without resulting in a change in the basic function to which it is related.

A multifunction valvuloplasty catheter device designed for positioning a guidewire to cross the aortic valve, atraumatic advancement and positioning of the device in the left ventricle, measurement of hemodynamics, and then aortic valvuloplasty without the need for catheter exchanges is provided.

In one embodiment, the device comprises a central guidewire lumen, a valvuloplasty balloon element, a shaped portion of the catheter disposed distal to the balloon adapted to position the guidewire so as to cross the stenotic aortic valve, a side hole for accurate hemodynamic pressure measurement, and an atraumatic tip adapted to minimize or reduce trauma within the left ventricle.

One method for using the device is to advance the device to the aortic valve, utilize the shaped portion of the catheter to facilitate crossing of the aortic valve, cross the aortic valve with a guidewire, advance the device over the guidewire to the left ventricle, remove the guidewire, measure hemodynamics via the central lumen, replace the guidewire, then perform balloon valvuloplasty by inflating and deflating the balloon portion of the device. If desired, the guidewire can then be removed to measure hemodynamics through the central lumen and assess the hemodynamic effect of the valvuloplasty.

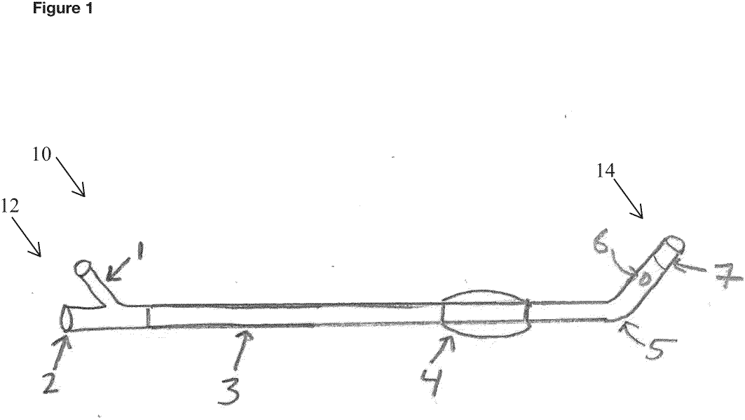

FIG. 1 is a schematic diagram of a multifunction valvuloplasty catheter device 10 adapted to position a guidewire to cross the aortic valve, atraumatic advancement and positioning of the device in the left ventricle, measurement of hemodynamics, and aortic valvuloplasty. In this embodiment, the multifunction valvuloplasty catheter device 10 comprises a proximal end 12 and a distal end 14. The proximal end of the catheter has two ports, such as with luer connections 1, 2. The first port comprises a central lumen 2 of the device to allow for guidewire passage. The second port comprises a second lumen 1 to allow for fluid to pass for balloon inflation and deflation. A valvuloplasty balloon 4 comprises a wrapped and pleated material set back from the distal end of the catheter and is in fluid communication with the balloon inflation catheter lumen to allow for balloon inflation and deflation. Multiple diameters and lengths of balloon may be designed to accommodate different aortic valve sizes and anatomies. Distal to the valvuloplasty balloon catheter, the catheter has a shaped curve portion 5 adapted to optimize or improve guidewire positioning by directing the guide wire towards the orifice of the aortic valve to facilitate guidewire crossing of the valve. The shaped portion of the catheter may be designed in multiple shapes as demonstrated in the example embodiments shown in FIGS. 1, 2, and 3 to accommodate different anatomic variants. The distal tip 7 of the catheter is constructed of a soft material different from the material of the rest of the catheter shaft 3 to be positioned atraumatically within the left ventricle. The distal tip has a lumen to allow passage of a guidewire. Distal to the balloon 4, at least one side hole 6 is defined within the catheter shaft and extends through a sidewall of the catheter shaft 3 to the central lumen to facilitate accurate hemodynamic measurement.

In the embodiment shown in FIG. 1, for example, the distal end 14 of the catheter 10 has a single bend or curvature of about 75 degrees. That provides a generally bent tip adapted to direct the guide wire through the aortic valve orifice and into an open region of the left ventricle of a patient’s heart.

In one embodiment, the shaped portion 5 of the catheter 40 is deflectable to actively change the shape of the catheter via a tensioning and/or stiffening device disposed along the catheter shaft. This can be achieved with a braided wire reinforced, coil wire reinforced, or laser-cut hypotube reinforced catheter design with one or multiple pull wires attached to anchor rings along the length of the catheter to deflect the shape of the catheter shaft and/or change the direction of the tip of the catheter.

In one embodiment, the shaft of the catheter is a made of a flexible, kink resistant material such as a thermoelastic polymer. This can be enhanced with a wire braided or coil braided design to enhance pushability, torque control, and kink resistance. The lumen of the catheter is designed to accommodate guidewires such as commonly used 0.035” and/or 0.038” diameter guidewires. The balloon material can be made in a semi compliant design to optimize profile and deliverability. Sample materials for semi compliant balloons include thermoelastic polymer or high durometer polyurethanes. The balloon material can also be made in a noncompliant design to achieve higher inflation pressures, resistant to rupture with more precise sizing of expansion. Sample materials for noncompliant balloon material include polyester and nylon. Dimensions of the balloon portion would be 3-5 cm in length with a range of diameters such as 12 mm to 28 mm for adults. The tip of the catheter is a soft flexible portion approximately 1-5 mm in length that could be made of a softer polymer. This portion would be low profile to be able to cross the stenotic aortic valve orifice and would be soft so as to not cause trauma to the left ventricle when it is placed therein.

Several processes of hemodynamic measurement are described here. After the tip of the multifunction valvuloplasty catheter device is placed in the left ventricle the guidewire is removed from the central lumen. The central lumen is then attached to tubing via a luer connection to a column of fluid in continuity with a pressure transducer. The pressure of the left ventricular cavity can be measured and recorded over time through the cardiac cycle. After the pressure measurement is recorded, the transducer is disconnected and the guide wire is replaced in the central lumen to continue with the procedure. An alternative method is to have a miniaturized electronic pressure transducer mounted between the valvuloplasty portion of the device and the tip of the catheter. This could be attached to a recording device via an electrical connection at the proximal portion of the catheter so as to record pressures. This would permit pressure measurement without having to remove the guidewire or attach tubing to the central lumen. An alternative method is to have a miniaturized fiber optic pressure transducer mounted between the valvuloplasty portion of the device and the tip of the catheter. This could be attached to a recording device via an optical connection at the proximal portion of the catheter so as to record pressures.

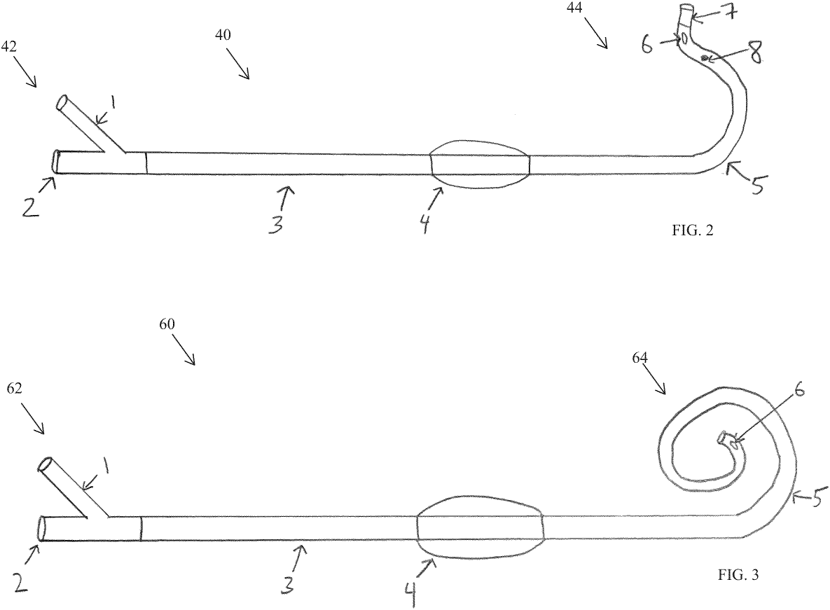

FIG. 2 is a schematic diagram of another representative embodiment of a multifunction valvuloplasty catheter device 40 with a different shaped catheter adapted to cross the aortic valve. In this embodiment, the multifunction valvuloplasty catheter device 40 is again adapted to position a guidewire to cross the aortic valve, atraumatic advancement and positioning of the device in the left ventricle, measurement of hemodynamics, and aortic valvuloplasty. In this embodiment, the multifunction valvuloplasty catheter device 40 comprises a proximal end 42 and a distal end 44. The proximal end of the catheter has two ports, such as with luer connections. The first port comprises a central lumen 2 of the device to allow for guidewire passage. The second port comprises a second lumen 1 to allow for fluid to pass for balloon inflation and deflation. A valvuloplasty balloon 4 comprises a wrapped and pleated material set back from the distal end of the catheter and is in fluid communication with the balloon inflation catheter lumen to allow for balloon inflation and deflation. Multiple diameters and lengths of balloon may be designed to accommodate different aortic valve sizes and anatomies. Distal to the valvuloplasty balloon catheter, the catheter has a shaped curve portion 5 adapted to optimize or improve guidewire positioning at the aortic valve and to facilitate the guidewire crossing the aortic valve orifice. The distal tip 7 of the catheter is constructed of a soft material different from the material of the rest of the catheter shaft 3 so as to be positioned atraumatically within the left ventricle. The distal tip 7 has a lumen to allow passage of a guidewire. Distal to the balloon 4, at least one side hole 6 is defined within the catheter shaft and extends through a sidewall of the catheter shaft 3 to the central lumen to facilitate accurate hemodynamic measurement.

In the embodiment shown in FIG. 2, for example, the distal end 44 of the catheter 40 has a double bend with a primary curvature of approximately 180 degrees with a distal secondary to the opposite direction of approximately 90 degrees. That provides a generally S-shaped bent tip adapted to curve through the aortic valve and into an open region of the left ventricle of a patient’s heart.

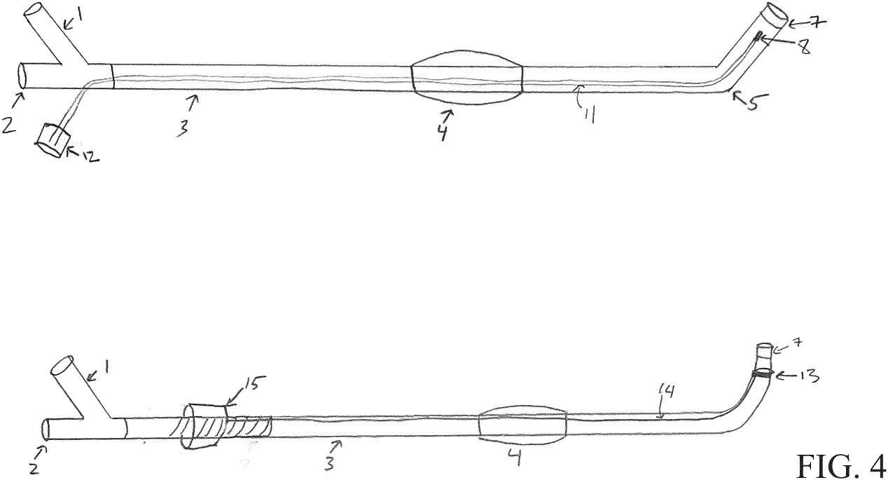

In one embodiment, the shaped portion 5 of the catheter 40 is deflectable to actively change the shape of the catheter via a tensioning and/or stiffening device disposed along the catheter shaft. This can be achieved with a braided wire reinforced, coil wire reinforced, or laser-cut hypotube reinforced catheter design with one or multiple pull wires attached to anchor rings along the length of the catheter to deflect the shape of the catheter shaft and/or change the direction of the tip of the catheter. In an example of the embodiment shown in FIG. 4, a knob 15 on the proximal portion of the shaft engages helical threads to pull a cable 14 traveling in the shaft attached to an anchor ring 13. By tensioning the cable the tip of the catheter becomes deflected.

FIG. 3 is a schematic diagram of another representative embodiment of a multifunction valvuloplasty catheter device 60 with a pigtail-shaped catheter adapted to cross the aortic valve. In this embodiment, the multifunction valvuloplasty catheter device 60 is again adapted to position a guidewire to cross the aortic valve, atraumatic advancement and positioning of the device in the left ventricle, measurement of hemodynamics, and aortic valvuloplasty. In this embodiment, the multifunction valvuloplasty catheter device 40 comprises a proximal end 62 and a distal end 64. The proximal end of the catheter has two ports, such as with luer connections. The first port comprises a central lumen 2 of the device to allow for guidewire passage. The second port comprises a second lumen 1 to allow for fluid to pass for balloon inflation and deflation. A valvuloplasty balloon 4 comprises a wrapped and pleated material set back from the distal end of the catheter and is in fluid communication with the balloon inflation catheter lumen to allow for balloon inflation and deflation. Multiple diameters and lengths of balloon may be designed to accommodate different aortic valve sizes and anatomies. Distal to the valvuloplasty balloon catheter, the catheter has a pigtail-shaped curve portion 5 adapted to optimize or improve guidewire positioning at the aortic valve and to facilitate guidewire crossing the aortic valve orifice. The distal tip 7 of the catheter may be constructed of a soft material different from the material of the rest of the catheter shaft 3 to be positioned atraumatically within the left ventricle, but due to the inherent atraumatic nature of the pigtail shape it may be constructed of the same material as the catheter shaft. The distal tip has a lumen to allow passage of a guidewire. Distal to the balloon 4, at least one side hole 6 is defined within the catheter shaft and extends through a sidewall of the catheter shaft 3 to the central lumen to facilitate accurate hemodynamic measurement.

In the embodiment shown in FIG. 3, for example, the distal end 64 of the catheter 60 has a pigtail-shaped bend or curvature of about 450 degrees. That provides a generally pigtail-shaped bent tip adapted to curve through the aortic valve and into an open region of the left ventricle of a patient’s heart. The curvature of the pigtail-shaped tip prevents the tip of the distal end from contacting the wall of the left ventricle.

In one embodiment, the shaped portion 5 of the catheter 40 is deflectable to actively change the shape of the catheter via a tensioning and/or stiffening device disposed along the catheter shaft. This can be achieved with a braided wire reinforced, coil wire reinforced, or laser-cut hypotube reinforced catheter design with one or multiple pull wires attached to anchor rings along the length of the catheter to deflect the shape of the catheter shaft and/or change the direction of the tip of the catheter.

FIG. 4 is a schematic diagram of another representative embodiment of a multifunction valvuloplasty catheter device 80 similar in design to the catheter device 10 shown in FIG. 1. In the embodiment of FIG. 4, however, the at least one hole/opening 6 of the catheter device 10 is replaced with at least one pressure sensor 8. An alternative method is to have a miniaturized electronic pressure transducer mounted between the valvuloplasty portion of the device and the tip of the catheter. Electrically conducting wires 11 would run along the length of the shaft. This could be attached to a recording device via an electrical connector 12 at the proximal portion of the catheter so as to record pressures. This would permit pressure measurement without having to remove the guidewire or attach tubing to the central lumen. An alternative method is to have a miniaturized fiber optic pressure transducer mounted between the valvuloplasty portion of the device and the tip of the catheter. This could be attached to a recording device via an optical connection at the proximal portion of the catheter to record pressures. The pressure sensor is electrically, optically, or mechanically coupled to a controller. The controller is adapted to receive at least one signal from the pressure sensor 8 to determine a hemodynamic measurement. This device would be optimized to measure left ventricular pressures through the cardiac cycle to provide the aortic valve pressure gradient and the left ventricular end diastolic pressure. The aortic valve pressure gradient is the difference in pressure between the left ventricle and the aortic pressures, and reflects the degree of aortic valve stenosis. The left ventricular end diastolic pressure is a measurement of the filling pressure and loading conditions of the left ventricle.

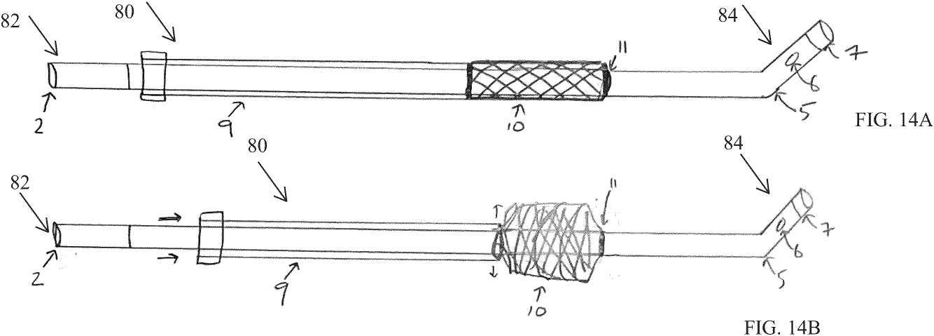

In another embodiment, the valvuloplasty function of the multifunction catheter 80 is accomplished by mechanical expansion device disposed between a proximal end 82 and a distal end 84 of the catheter 80. In an example of the embodiment demonstrated in FIGS. 14A and 14B, the central lumen shaft is surrounded by multiple interwoven metallic or plastic wires 10 which are fixed to the shaft at the distal most portion 11. An example interwoven material would be nitinol wires. The proximal portion of the interwoven wires are attached to an outer shaft 9 surrounding the central lumen. By advancing the outer shaft towards the distal end 84 of the catheter, the nitinol wires are compressed in length and expand in width. This expansion in width within the aortic valve would be adequate to perform aortic valvuloplasty. One example mechanical expansion device adapted for use in a valvuloplasty procedure comprises an expandable braid valvuloplasty device, such as described in U.S. Pat. no. 10,716,663 entitled “Methods and Apparatus for Performing Valvuloplasty,” which is incorporated by reference as if fully set forth herein.

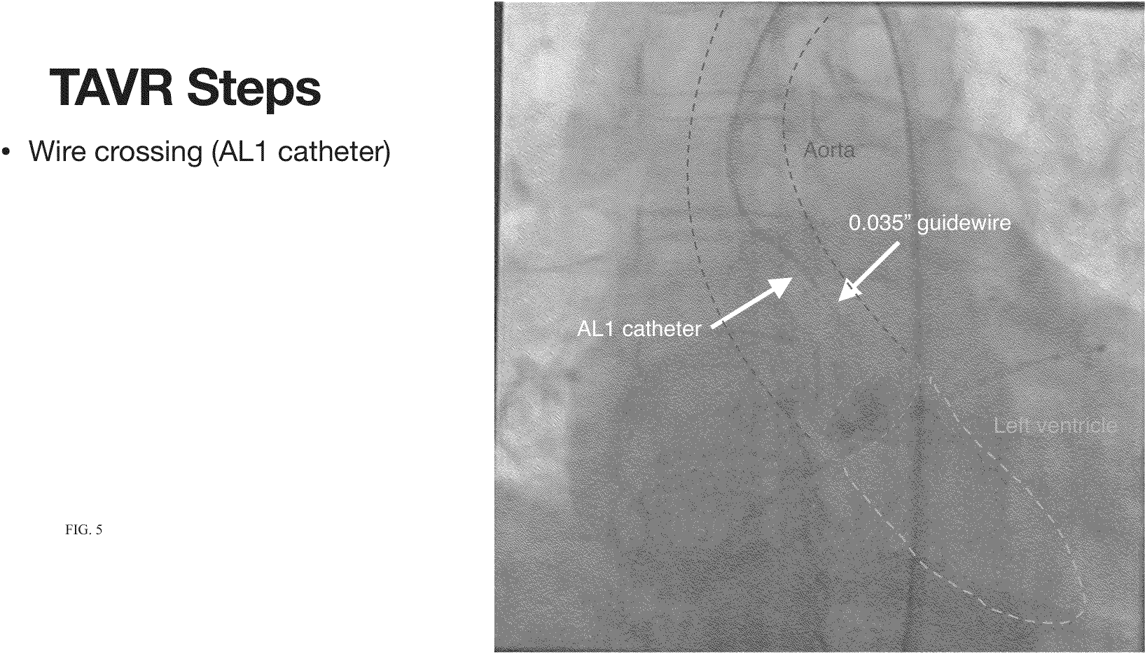

FIGS. 5-13 show steps of an example transcatheter aortic valve replacement procedure (TAVR) procedure. This multifunction catheter may be used as part of a TAVR procedure, or as a standalone balloon aortic valvuloplasty not accompanied by TAVR, or as part of another procedure.

In FIG. 5, for example, a guidewire (e.g., a 0.035” guidewire) and a catheter are extended within the aorta approaching the aortic valve of a patient’s heart leading to the left ventricle. This may be accomplished by extending the guide wire to the aortic valve, fixing the wire in place, and then advancing the catheter over the fixed wire, or by advancing the tip of the wire beyond (e.g., a few cm beyond) the tip of the catheter and then advancing the two en bloc to the aortic valve.

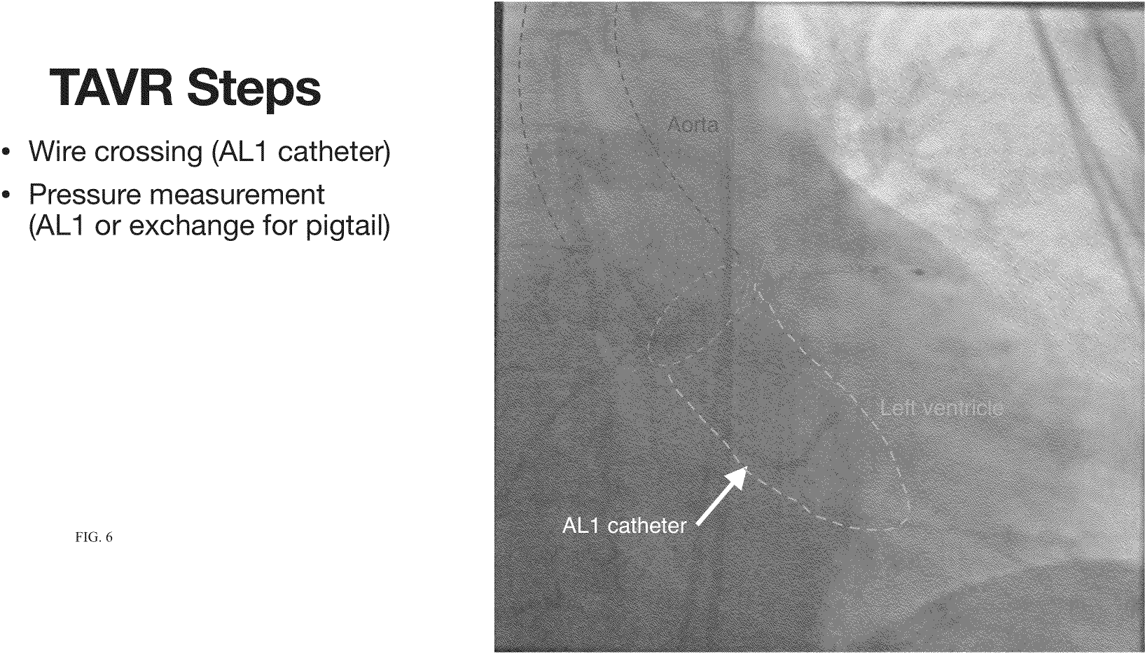

In FIG. 6, the guidewire and catheter are extended through the aortic valve into the left ventricle, and the guidewire is withdrawn from the catheter. The end of the catheter is extended into the left ventricle and a hemodynamic measurement (e.g., pressure measurement) is taken using at least one opening in the catheter or pressure sensor disposed in the catheter.

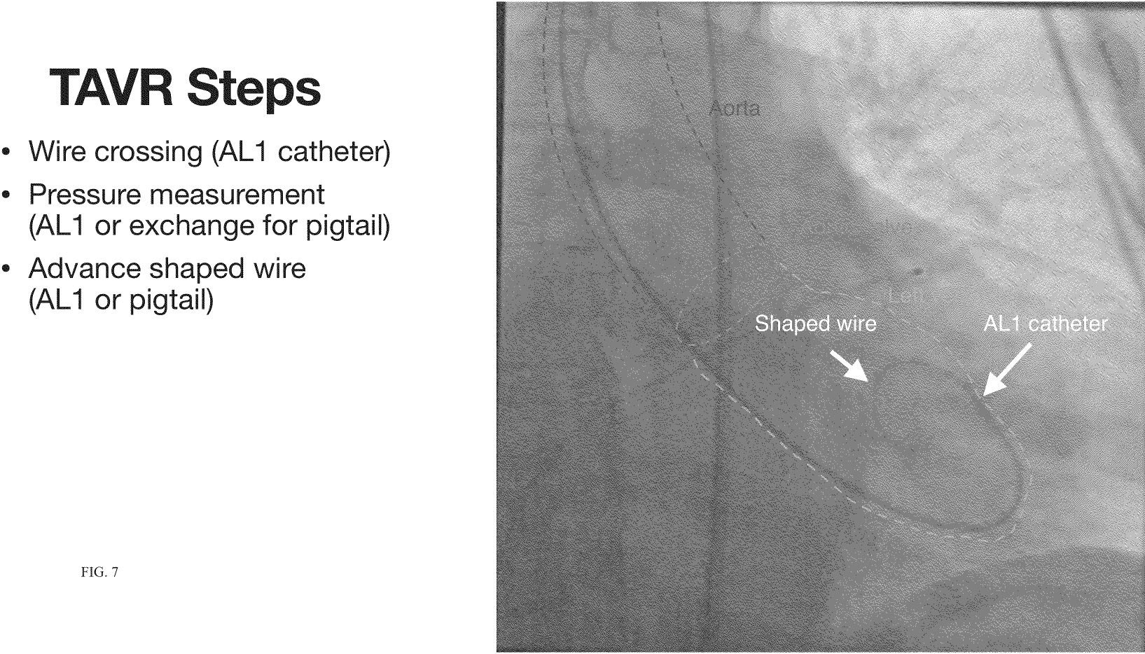

In FIG. 7, the guidewire is extended back through the catheter and extends into the left ventricle. In this example, the guidewire comprises a generally pigtail shaped distal end in which the distal tip of the guidewire is disposed within a pigtail loop that prevents the distal tip from contacting a wall of the left ventricle.

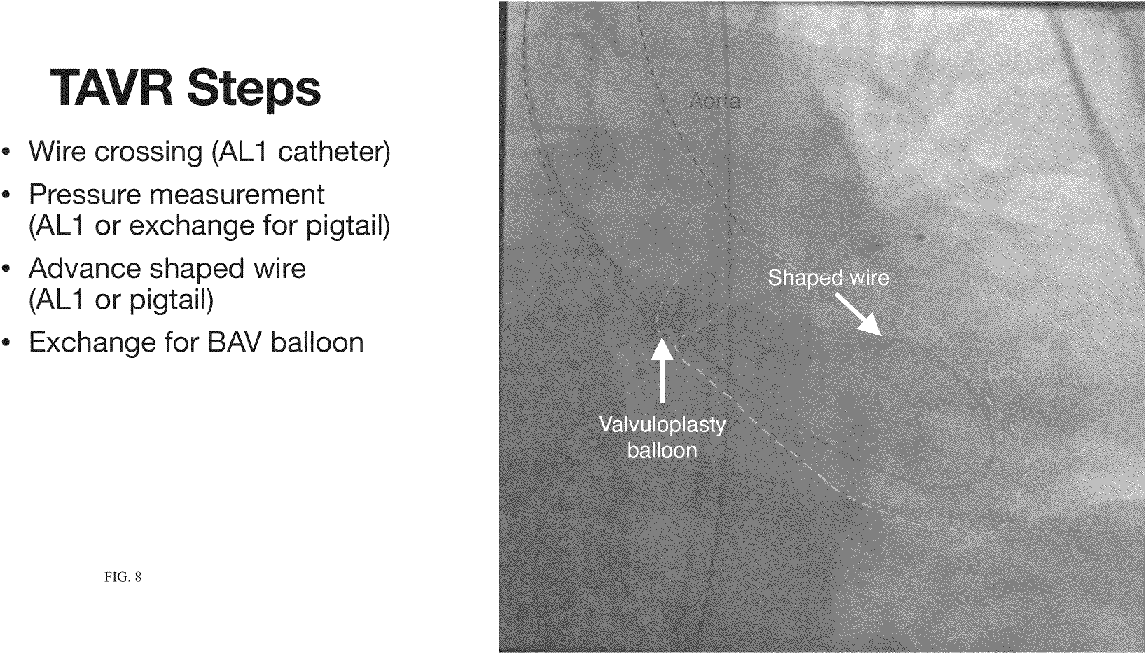

In FIG. 8, the catheter balloon is aligned with the aortic valve.

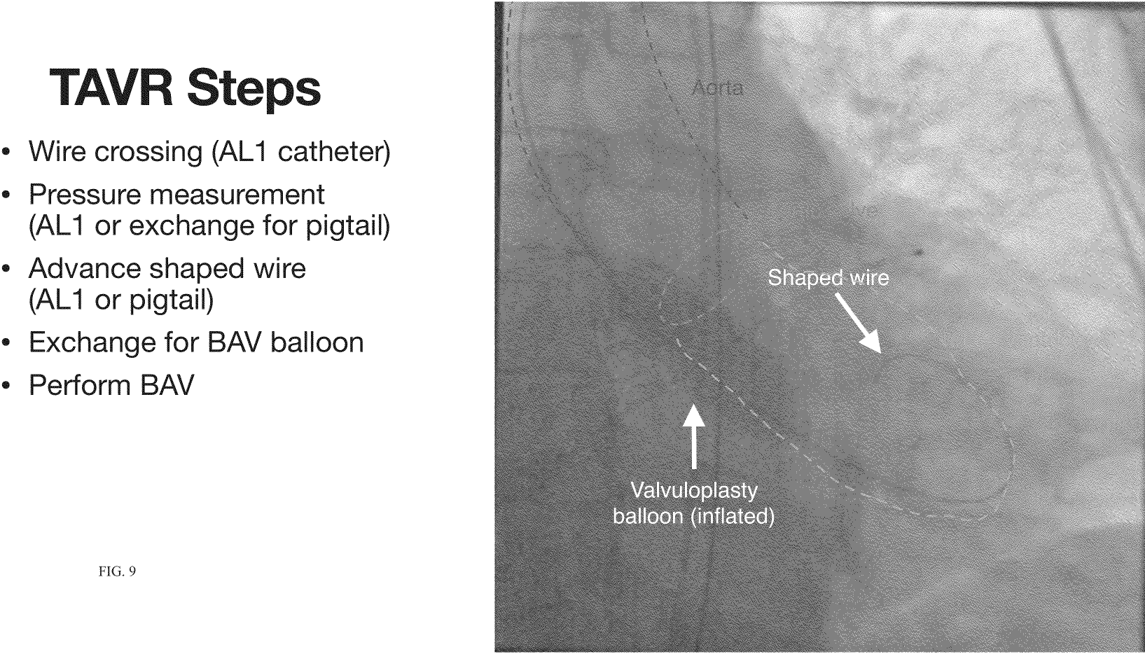

In FIG. 9, the balloon is inflated within the aortic valve to perform a valvuloplasty procedure. The shaped guidewire distal end is in a generally pigtail configuration in which the distal tip of the guidewire is disposed within a pigtail loop that prevents the distal tip from contacting a wall of the left ventricle.

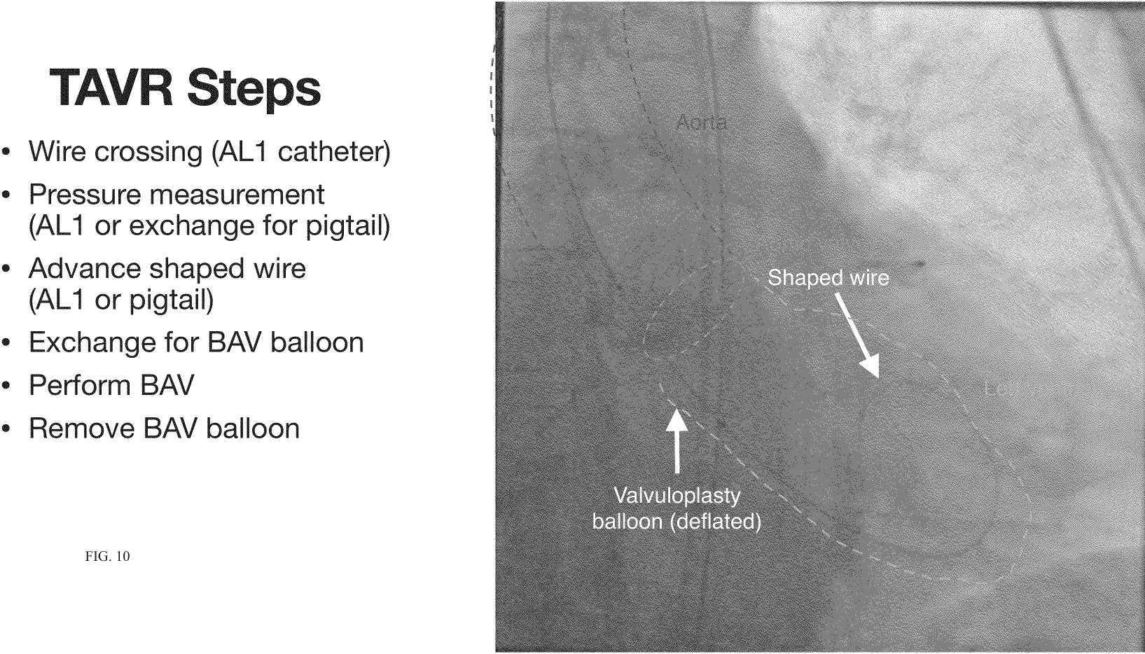

In FIG. 10, the balloon of the catheter is deflated within the aortic valve in the valvuloplasty procedure. The inflation and deflation operations may be repeated one or more times as desired.

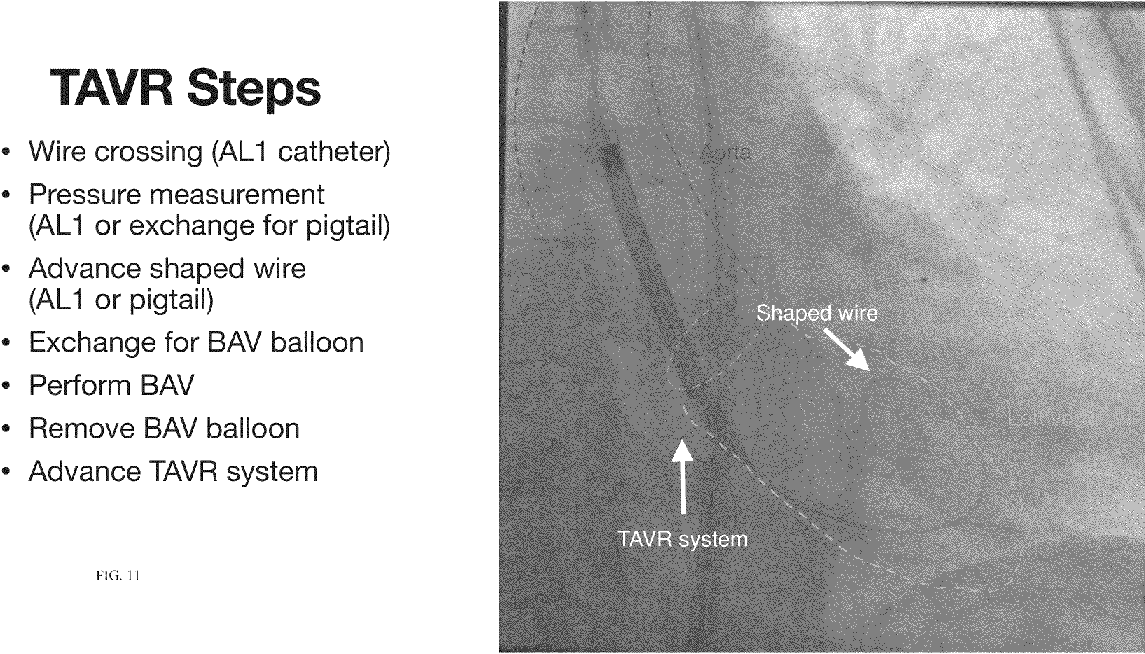

In FIG. 11, the valvuloplasty catheter device is withdrawn along the guidewire and a TAVR system is advanced along the guidewire to the aortic valve.

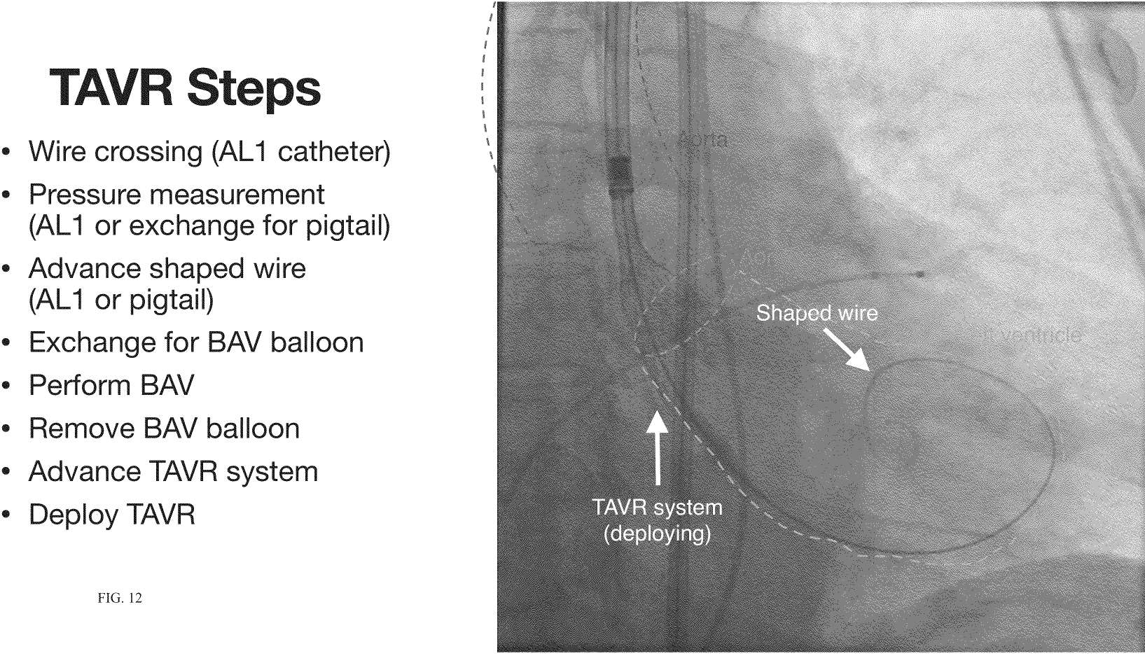

The TAVR system is deployed in FIG. 12 at the aortic valve.

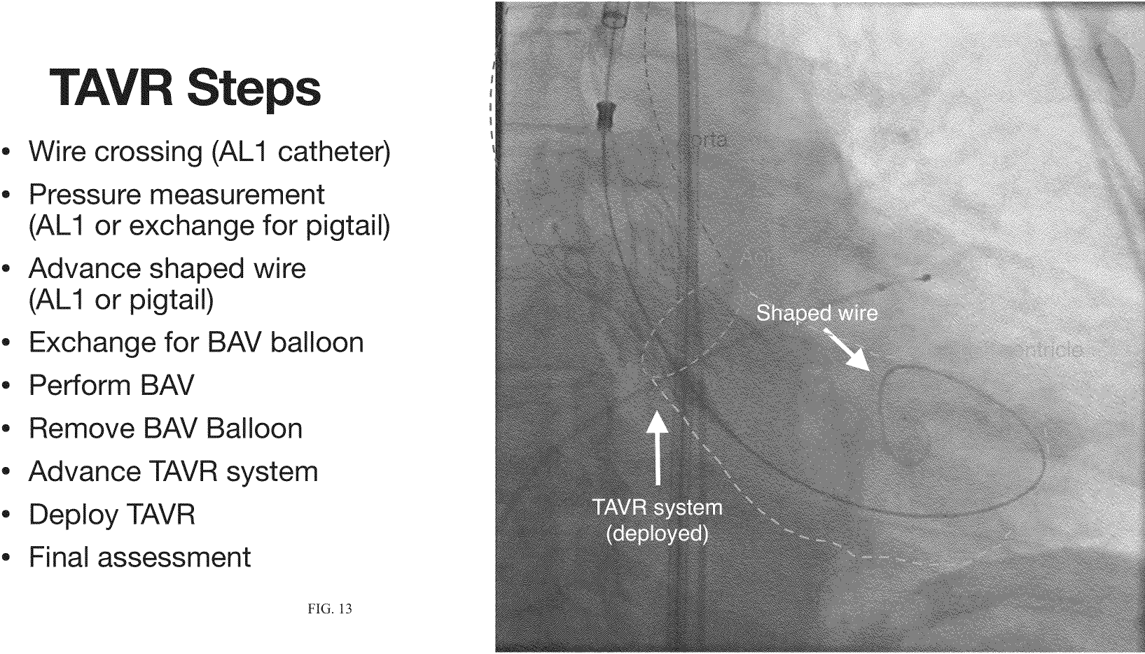

In FIG. 13, a final assessment of the TAVR procedure is performed. This can be achieved by one or more of the following: clinical assessment, hemodynamic pressure monitoring, echocardiographic assessment, or aortic root angiography.

In the example operation shown in FIGS. 5-13, a plurality of operations of a typical TAVR procedure are performed by the multifunction valvuloplasty catheter device instead of a plurality of typical devices needed to perform the procedure. In particular, the multifunction valvuloplasty catheter device is adapted to perform the following operations:
wire crossing through the aorta and aortic valve into the left ventricle;
pre-procedure pressure measurement within the left ventricle;
advance of a shaped wire into the left ventricle;
deployment of a balloon within the aortic valve;
inflation of the balloon within the aortic valve;
deflation of the balloon within the aortic valve;
removal of the balloon from the aortic valve; and
post-procedure pressure measurement within the left ventricle.

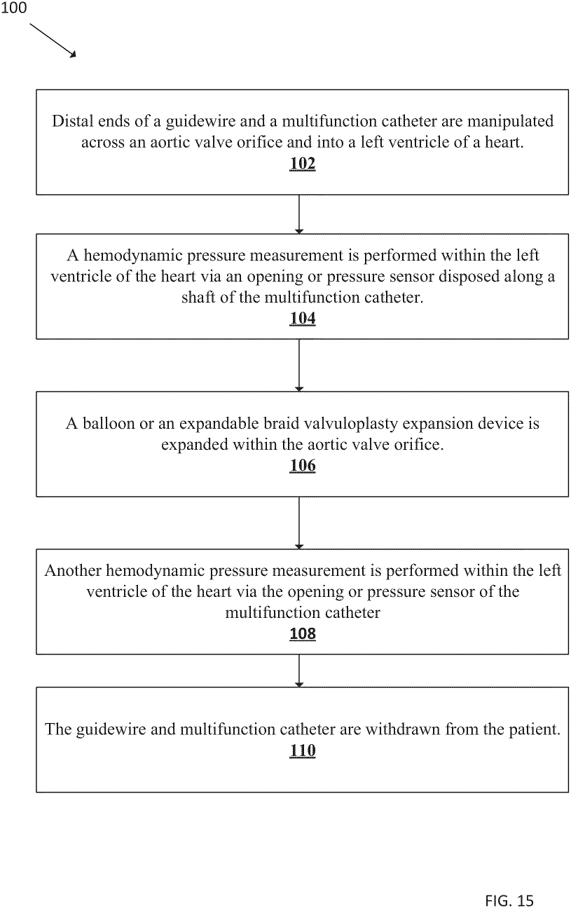

FIG. 15 is a flow chart showing operations of an example method 100 of using a multifunction catheter as described herein. As shown in FIG. 15, distal ends of a guidewire and a multifunction catheter are manipulated across an aortic valve orifice and into a left ventricle of a heart in operation 102. In various aspects, the guidewire and catheter may be inserted into the arterial system of a patient together and directed to the aortic valve orifice, or the guidewire may be inserted into the patient arterial system first, and then the catheter is directed along the guidewire to the aortic valve orifice.

In operation 104, a hemodynamic pressure measurement is performed within the left ventricle of the heart via an opening or pressure sensor disposed along a shaft of the multifunction catheter. A balloon or an expandable braid valvuloplasty expansion device is expanded within the aortic valve orifice in operation 106. The balloon or the expandable braid valvuloplasty expansion device is disposed proximal to the opening or pressure sensor along the shaft of the multifunction catheter.

Another hemodynamic pressure measurement is performed within the left ventricle of the heart via the opening or pressure sensor of the multifunction catheter in operation 108. It should be noted that neither operation 104 and 108, one of operation 104 and 108, or both operation 104 and 108 may be performed in various implementations.

The guidewire and multifunction catheter are withdrawn from the patient in operation 110. Again, the guidewire and catheter may be withdrawn together or separately.

What is claimed is:

1. A multifunction catheter adapted for valvuloplasty, the multifunction catheter comprising:
a shaft defining at least one lumen, the shaft comprising a distal end, a proximal end, and a bend;
a valvuloplasty expansion device comprising a balloon or expandable braid valvuloplasty device, the valvuloplasty expansion device disposed along the shaft between the proximal end and the bend of the shaft; and
an opening through the shaft or a pressure sensor, the opening or pressure sensor adapted to facilitate a hemodynamic pressure measurement within a left ventricle of a heart and the opening or pressure sensor is disposed along the shaft between the bend and the distal end of the shaft.

2. The multifunction catheter of claim 1, wherein the bend of the shaft comprises at least one of the group comprising: a single bend, a double bend, and a pigtail bend.

3. The multifunction catheter of claim 2, wherein the single bend comprises a bend of approximately 75 degrees.

4. The multifunction catheter of claim 2, wherein the double bend comprises a primary curvature of approximately 180 degrees with a distal secondary to the opposite direction of approximately 90 degrees.

5. The multifunction catheter of claim 2, wherein the pigtail bend comprises a bend of approximately 450 degrees.

6. The multifunction catheter of claim 1, wherein the pressure sensor comprises at least one of an electrical and mechanical pressure sensor adapted to facilitate a hemodynamic measurement within a left ventricle of a heart.

7. The multifunction catheter of claim 1, wherein the opening comprises a plurality of openings.

8. The multifunction catheter of claim 1, wherein an atraumatic tip is disposed at the distal end of the shaft.

9. The multifunction catheter of claim 1, wherein the balloon comprises a wrapped and pleated material disposed along a length of the shaft.

10. The multifunction catheter of claim 1, wherein at least one port is disposed in fluid communication with the at least one lumen.

11. The multifunction catheter of claim 1, wherein a pair of luer ports are disposed between the proximal end of the shaft and the balloon, each of the pair of luer ports is in fluid communication with the at least one lumen.

12. The multifunction catheter of claim 1, wherein a pair of luer ports are disposed between the proximal end of the shaft and the balloon, each of the pair of luer ports is in fluid communication with each of a pair of lumens disposed within the shaft.

13. The multifunction catheter of claim 1, wherein the valvuloplasty expansion device comprises an expandable braid valvuloplasty device, the expandable braid valvuloplasty device comprising a plurality of interwoven wires surrounding the at least one lumen defined by the shaft, the plurality of interwoven wires are fixed to the shaft distal to an expansion location of the valvuloplasty expansion device.

14. The multifunction catheter of claim 13, wherein the plurality of interwoven wires comprises nitinol wires.

15. The multifunction catheter of claim 13, wherein the plurality of interwoven wires comprises metallic or plastic wires.

16. The multifunction catheter of claim 13, wherein a proximal portion of the plurality of interwoven wires are attached to an outer shaft surrounding the central lumen, the plurality of interwoven wires are adapted to expand in width and compress in length as the outer shaft is advanced toward the distal end of the shaft.

17. A method for performing a valvuloplasty procedure on a patient, the method comprises:
crossing distal ends of a guidewire and a multifunction catheter across an aortic valve orifice and into a left ventricle of a heart;
performing a hemodynamic pressure measurement within the left ventricle of the heart via an opening or pressure sensor disposed along a shaft of the multifunction catheter;
expanding a balloon or an expandable braid valvuloplasty expansion device within the aortic valve orifice, the balloon or the expandable braid valvuloplasty expansion device is disposed proximal to the opening or pressure sensor along the shaft of the multifunction catheter; and
withdrawing the guidewire and multifunction catheter from the patient.

18. The method of claim 17, wherein the multifunction catheter comprises:
a shaft defining at least one lumen, the shaft comprising a distal end, a proximal end, and a bend;
a valvuloplasty expansion device comprising a balloon or expandable braid valvuloplasty device, the valvuloplasty expansion device disposed along the shaft between the proximal end and the bend of the shaft; and
an opening through the shaft or a pressure sensor, the opening or pressure sensor adapted to facilitate a hemodynamic pressure measurement within a left ventricle of a heart and the opening or pressure sensor is disposed along the shaft between the bend and the distal end of the shaft.

19. The method of claim 17, wherein a second hemodynamic pressure measurement is performed after expanding the balloon or the expandable braid valvuloplasty expansion device within the aortic valve orifice.

20. The method of claim 17, wherein the guidewire and the multifunction catheter are advanced through the aortic valve orifice together.

21. The method of claim 17, wherein the guidewire is advanced through the aortic valve orifice followed by the multifunction catheter being advanced along the guidewire.
